SEQUENCE LISTING (2 sequences)

SEQ ID NO: 1 - DNA, 1538 bp, Homo sapiens
ggacttccggcgggtgacgcgcccgggttcggctacaaaagaggacggctgcggcgcgccgggcggaactttccagaacgctcggtgagaggcggaggagcggtaactaccccggctgcgcacagctcggcgctccttcccgctccctcacacaccggcctcagcccgcaccggcagtagaagatggtgaaagaaacaacttactacgatgttttgggggtcaaacccaatgctactcaggaagaattgaaaaaggcttataggaaactggctttgaagtaccatcctgataagaacccaaatgaaggagagaagtttaaacagatttctcaagcttacgaagttctctctgatgcaaagaaaagggaattatatgacaaaggaggagaacaggcaattaaagagggtggagcaggtggcggttttggctcccccatggacatctttgatatgttttttggaggaggaggaaggatgcagagagaaaggagaggtaaaaatgttgtacatcagctctcagtaaccctagaagacttatataatggtgcaacaagaaaactggctctgcaaaagaatgtgatttgtgacaaatgtgaaggtagaggaggtaagaaaggagcagtagagtgctgtcccaattgccgaggtactggaatgcaaataagaattcatcagataggacctggaatggttcagcaaattcagtctgtgtgcatggagtgccagggccatggggagcggatcagtcctaaagatagatgtaaaagctgcaacggaaggaagatagttcgagagaagaaaattttagaagttcatattgacaaaggcatgaaagatggccagaagataacattccatggtgaaggagaccaagaaccaggactggagccaggcgatattatcattgtgttagatcagaaggaccatgctgtttttactcgacgaggagaagaccttttcatgtgtatggacatacagctcgttgaagcactgtgtggcttccagaagccaatatctactcttgacaaccgaaccatcgtcatcacctctcatccaggtcagattgtcaagcatggagatatcaagtgtgtactaaatgaaggcatgccaatttatcgtagaccatatgaaaagggtcgcctaatcatcgaatttaaggtaaactttcctgagaatggctttctctctcctgataaactgtctttgctggaaaaactcctacccgagaggaaggaagtggaagagactgatgagatggaccaagtagaactggtggactttgatccaaatcaggaaagacggcgccactacaatggagaagcatatgaggatgatgaacatcatcccagaggtggtgttcagtgtcagacctcttaatgggccagtgaataacactcactgctggcatttaatgtgcagtagtgaatgagtgaaggactgtaatcataatatgctcactacttgctcttgtttttgttttaataaactatagtagtgttttaaaaagttaaatgaagaataaacgcaaatataaaagc

SEQ ID NO: 2 - protein, 397 aa, Homo sapiens
MVKETTYYDVLGVKPNATQEELKKAYRKLALKYHPDKNPNEGEKFKQISQAYEVLSDAKKRELYDKGGEQAIKEGGAGGGFGSPMDIFDMFFGGGGRMQRERRGKNVVHQLSVTLEDLYNGATRKLALQKNVICDKCEGRGGKKGAVECCPNCRGTGMQIRIHQIGPGMVQQIQSVCMECQGHGERISPKDRCKSCNGRKIVREKKILEVHIDKGMKDGQKITFHGEGDQEPGLEPGDIIIVLDQKDHAVFTRRGEDLFMCMDIQLVEALCGFQKPISTLDNRTIVITSHPGQIVKHGDIKCVLNEGMPIYRRPYEKGRLIIEFKVNFPENGFLSPDKLSLLEKLLPERKEVEETDEMDQVELVDFDPNQERRRHYNGEAYEDDEHHPRGGVQCQTS

(19) United States
(12) Patent Application Publication
(10) Pub. No.: US 2011/0045509 A1
(43) Pub. Date: Feb. 24, 2011

(54) Method For Determining The Type of an Inflammatory-Rheumatic Disease in Synovial Fluid

(76) Inventor: Inga Melchers, Schallstadt (DE)

Correspondence Address: CURATOLO SIDOTI CO., LPA, 24500 CENTER RIDGE ROAD, SUITE 280, CLEVELAND, OH, 44145, US

(21) Appl. No.: 12/933,475

(22) PCT Filed: Mar. 5, 2009
(86) PCT No.: PCT/EP2009/001569

§ 371 Date: Nov. 12, 2010

(30) Foreign Application Priority Data
Mar. 20, 2008 (EP) 08005272.3

(51) Int. Cl. G01N 33/53; G01N 33/68; C07K 16/00

(52) U.S. Cl. 435/7.92; 436/86; 436/501; 530/389.1

(57) ABSTRACT

Disclosed is an in vitro method for determining the type of an inflammatory-rheumatic disease, wherein the presence or absence of the protein Hdj2 in synovial fluid is determined at an early stage of the disease.

Arthritis is an inflammation of the joints which can be recognized by the following symptoms: pain, swelling, warmth, restricted movement, joint effusion, joint emphysema, sometimes redness. In chronic progressions of the disease, erosions which can change and destroy the joint can also occur. In the progression of the disease, misalignment, muscle shortening, and stiffening can occur, often affecting especially the small joints of the feet and hands.

There are very many different forms of arthritis, which can be categorized according to different criteria. For the prognosis and therapy of an arthritic disease, it is essential that the type of arthritis and the cause of this arthritis can be diagnosed as easily, early, and reliably as possible so that the most effective therapy can be selected as early as possible. About half of patients with undifferentiated arthritis develop a substantial restriction in function of the affected joints within the next 5 years, while the others show a mild progression, in part even without therapy. A prognosis of the likely further progression of the disease is therefore desirable.

Within the present invention, it was discovered that a certain protein having the designation Hdj2, which normally occurs only intracellularly, appears frequently, at an early stage, and in a considerable quantity in the synovial fluids of patients with rheumatoid arthritis (hereinafter RA), whereby a differentiation of patients with rheumatoid arthritis and other patients who have some other inflammatory rheumatic condition is made possible.

Hdj2, a normally intracellular J-protein, occurs significantly more frequently as an extracellular molecule in synovial fluids of patients with rheumatoid arthritis (RA) than in synovial fluids of patients with other arthritides (non-RA patients). Hdj2 in synovial fluid shows significant correlations with the appearance of two autoantibodies associated with RA (rheumatoid factor, anti-CCP). In RA patients, the Hdj2 quantity correlates positively with the degree of inflammation, measured as the number of infiltrating leukocytes. Hdj2 is strongly overexpressed in the synovial lining cells of patients with RA, but not in synovial tissue from patients with osteoarthritis.

The J-protein Hdj2 (DNAJA1, Hsj2, Hsdj, dj-2) is one of about 40 known human J-proteins. J-proteins have a highly conserved J-domain which is highly homologous to DnaJ of E. coli (they are therefore also called DnaJ-like proteins). J-proteins are considered to be co-chaperones of members of the HSP70 family, with which they interact via the J-domain and function as a chaperone machine. The already known messenger RNA is reproduced as a DNA sequence in the sequence protocol as SEQ ID NO:1.

The nucleic acid encodes the Hdj2 protein. The already known amino acid sequence is reproduced in the sequence protocol as SEQ ID NO:2.

Within the present invention, it was determined that, surprisingly, Hdj2 is useful as an early marker for the diagnosis of rheumatoid arthritis because, specifically in the early stages of the disease, an increased content of Hdj2 is detected in the synovial fluid of such patients who go on to develop rheumatoid arthritis. This is surprising in that previous investigations showed that Hdj2 is also detected in the synovial fluid of patients who develop various rheumatic diseases. In the present case, an important aspect of the present invention is that Hdj2 appears in synovial fluid at early disease stages in those patients who go on to develop rheumatoid arthritis. In patients who develop other forms of an inflammatory rheumatic disease, this marker, in contrast, appears only to a very small extent in synovial fluid, if at all.

Inflammatory rheumatic diseases often begin with painful swellings of the joints. In RA, so-called morning stiffness, with a duration of more than 1 hour, is a further early characteristic. Inflammatory polyarthritis, in which more than 3 joint regions are affected, including hand and finger joints, and which is symmetrical, could be early RA. The clinical signs of early RA must persist for at least 6 weeks in order to be able to make a diagnosis. Autoantibodies, such as rheumatoid factor and anti-CCP antibody, can be detectable in blood. These autoantibodies partly precede the symptoms, but can also appear later. In contrast, erosions and rheumatoid nodules generally develop with further progression of the disease. The extent of the swellings, the morning stiffness, and the pain can strongly fluctuate during the progression of the disease. Undifferentiated arthritis is still unexplained and not classified. Once a diagnosis has been made, the first 2-3 years can still be referred to as early (rheumatoid) arthritis. Patients with undifferentiated arthritis often exhibit only 2-3 of the ACR criteria, and the clinical signs of arthritis often persist for only 4 weeks. Thus, it is not possible to classify the patients as RA by means of the ACR criteria.

An early stage of the disease is herein understood to mean the interval which begins with the onset of rheumatic conditions and extends over a period of up to three years, preferably up to two years, more preferably up to one year, and particularly preferably up to six months. The onset of an inflammatory rheumatic condition is understood to mean the appearance of the first symptoms. The first symptoms include morning stiffness of a duration of more than one hour and the appearance of swelling of the joints. In the progression of an inflammatory rheumatic disease, arthritis appears in two or more joint regions, more particularly arthritis of hand or finger joints. Rheumatoid nodules generally appear much later.

A further symptom for the beginning of an inflammatory rheumatic disease can be when swelling in more than two joints persists over a period of more than six weeks. The exact date of the appearance of an inflammatory rheumatic condition is very difficult to determine precisely. The first symptoms are noticed subjectively by the patients, and can only rarely be dated precisely. In these cases, the first diagnosis of an inflammatory rheumatic disease is therefore defined as the date at which the early phase of the disease begins.

Hdj2 is useful as an early marker for RA, since it was detectable in synovial tissue (one patient, see example 1) and in synovial fluid (several patients, see table 1) prior to making this diagnosis. It is useful as a marker for a differential diagnosis of RA, since it was present in persons with doubtful diagnoses who later did indeed or instead receive the diagnosis RA (table 1). Hdj2 was at the time point of diagnosis found in the synovial fluid of patients with RA, who, by fulfilling ≧4 of the 7 ACR criteria, were confidently classified as RA (table 2). Furthermore, Hdj2 was at the time of diagnosis not found in the synovial fluid of patients who were not classified as RA (table 2). In synovial liquids of patients with early arthritis (not more than 2 years after first diagnosis), there was a similar observation, i.e., Hdj2 was present in patients with RA, but not in patients with another diagnosis (corresponding data are summarized in table 3).

Rheumatoid arthritis is not only a disease of two or more joints, but a disease affecting the whole body. Rheumatoid arthritis is the most common form of joint inflammation, which, untreated, leads to destruction of the joints, to premature incapacity for work, and—since it is a systemic disease and inner organs are also affected, e.g., by endothelial dysfunction—to increased morbidity.

In rheumatoid arthritis, there is a derailment of the normal inflammation processes in the joints. The actual trigger of this disease has still not been identified to date, but rheumatoid arthritis is included among the autoimmune diseases. The pathogenesis is still not understood. It is believed that both genetic and environmental factors play a role; thus, it could be a multifactorial disease. Since relatives of a patient generally carry a higher risk of also contracting rheumatoid arthritis, it is believed that there could be genetic causes. However, to date, only a few genes or alleles are known which are associated with rheumatoid arthritis and affect a larger group of patients.

According to the current state of knowledge, the genetic association most important for rheumatoid arthritis involves the very polymorphous HLA complex. Persons who carry one or two HLA-DRB1 alleles which have an amino acid motif known as a “shared epitope” have a higher risk of contracting the disease than persons without this motif; however, there are also many healthy individuals with the “shared epitope” (SE). This motif consists of 5 amino acids and can be, in one-letter code, QKRAA, QRRAA, or RRRAA. In the Caucasian population of Europe and North America, QKRAA is the most common SE.

HLA-DRB1 genes encode HLA-DR molecules which play an important role as MHC class II molecules in the immune response. These molecules present, on the surfaces of antigen-presenting cells, peptides which can originate from antigens (e.g., in the case of an infection) or also from autoantigens (e.g., in the case of an autoimmune disease). Subsequently, T lymphocytes can be activated, which in turn help B lymphocytes to synthesize antibodies against the antigens or autoantigens. The appearance of autoantibodies which react with autoantigens represents a characteristic of an autoimmune response. RA can also be considered as an autoimmune disease because of the genetic association with HLA-DRB1 alleles and because of the appearance of autoantibodies (rheumatoid factor, which recognizes human IgG, and, recently, a further class of autoantibodies which are directed against citrullinated proteins and can be summarized here as anti-CCP).

Interestingly, then, the “shared epitope” was found not only on HLA-DR molecules; the sequence QKRAA also occurs in many bacteria. A prototype for it is E. coli (gut bacteria) and its DnaJ protein. This carries a J-domain in which the motif QKRAA of the SE is located. Furthermore, it was shown that the SE in DnaJ is important for the interaction with the partner molecule of DnaJ, DnaK (E. coli). DnaK is a heat shock protein (HSP) of the HSP70 family. There are HSP70 proteins in all organisms and, likewise, the J-proteins interacting with them (named after the J-domain of DnaJ). The HSP70 proteins and J-proteins together form a chaperone machine. They are thus involved in the correct folding of other proteins, such as, for example, becomes necessary after heat shock of a cell so that the cell can survive.

In humans, there are about 12 genes for HSP70 proteins and >40 genes for J-proteins. For many, only the nucleic acid sequence is known. The accordingly variedly assembled HSP70/J-protein chaperone machines contribute intracellularly to many functions of cells, not only in stress situations, but also in, for example, transport or protein synthesis. They are also involved in antigen presentation. The motif of the “shared epitope” is not found in the better known human J-proteins.

In the synovial tissue of patients with RA, at least one HSP70 molecule (Hsc70) and one J-protein (Hdj2) are very strongly overexpressed. It is known that both cooperate with one another and that Hsc70 is crucially involved in allele-specific antigen presentation in DRB1*0401-positive persons.

Without wishing to be tied to any one particular theory, it is believed within the present invention that an allele-specific antigen presentation is at the beginning of the disease. The molecules involved, including also possibly Hdj2, can thus be used as a marker for the development and appearance of rheumatoid arthritis. This marker can be used for a subgrouping of patients as a marker for differential diagnoses, monitoring disease progression, prognoses, and also for checking the effectiveness of the therapy. It was determined that, surprisingly the protein Hdj2, a protein normally occurring intracellularly, appears extracellularly in synovial fluid and makes possible a direct correlation to the classification of the inflammatory rheumatic disease. It is particularly surprising that this appearance takes place at a relatively early stage, making an early diagnosis possible.

At the beginning of arthritis, the joints often swell, and these joints are then aspirated as a therapeutic measure. The synovial fluid withdrawn is analyzed in order to obtain clues for the local condition and in order to receive information about the disease present. Physical and chemical properties, inflammation markers (C-reactive protein; the occurrence of leukocytes), and also possibly the occurrence of crystals (gout) or antibodies (e.g., rheumatoid factor) are investigated. Aspiration is carried out in very many inflammatory, but also noninflammatory, diseases.

At the beginning of arthritis, it is often not recognizable what type of arthritis it is (undifferentiated arthritis). There are many different forms, of which RA is only one. A good prognosis of future disease progression is desirable in order to be able to prevent serious consequences.

Currently, the so-called ACR criteria are used for diagnosing RA, wherein at least 4 of 7 possible criteria must be fulfilled in order to classify the disease as RA. However, this is often still not possible at the beginning of the disease. It is even more difficult to make an early prognosis about the further progression of RA, this being of great importance for the choice of therapy. Among the 7 ACR criteria, there is only one laboratory parameter (rheumatoid factor: anti-IgG). Lately there has been a second antibody (anti-CCP, anti-citrullinated-fibrinogen and other citrullinated proteins or peptides) which is also gaining increasing importance for diagnosing RA. Both autoantibodies overlap substantially; however, there are also RA patients who develop only one of the two or neither. Both autoantibodies also occur to a certain extent in other patients and healthy individuals. Although more particularly anti-CCP-antibodies are quite specific and appear relatively early, they are to be seen as a reaction to previously occurring changes in the individual. It is therefore desirable to find further parameters or markers which directly accompany these early changes and make possible a very early and reliable diagnosis and prognosis.

Like many other rheumatic diseases, RA is a disease which appears in many different forms. Currently, these are mainly divided into a seropositive and a seronegative form, owing to the presence or absence of rheumatoid factor. An anti-CCP-positive and an anti-CCP-negative form are also starting to be defined. Further classifications become potentially possible when further markers can be used and combinations of multiple markers also become possible. Genetic markers are currently not used at all.

For monitoring disease progression and for estimating individual risk, it is also desirable to have new markers, more particularly also those which can be found very early, i.e., preferably prior to the appearance of antibodies.

Since aspirates are solely withdrawn for clinical reasons, the synovial fluid can be considered as a “waste product” which can be used advantageously in diagnostic methods.

The present invention accordingly provides an in vitro method for classifying and prognosing a rheumatically inflammatory disease, wherein the presence or absence of the protein Hdj2 in synovial fluid is determined.

With the method, rheumatoid arthritis can be distinguished at an early stage from other rheumatic diseases, such as: infectious or septic arthritis, arthritis in rheumatic vertebral column diseases; arthritis in inflammatory gut diseases, juvenile chronic arthritis, arthritis in inflammatory connective tissue diseases; allergic arthritis; arthritis caused by metabolic diseases, gout, arthritis in endocrine disorders, such as hyperthyroidism, hypothyroidism, hyperparathyroidism, hypoparathyroidism, diabetes mellitus, Cushing's syndrome, acromegaly, pheochromocytoma; arthritis in granulomatous diseases; arthritis in joint bleeding as a result of disorders of blood coagulation, arthritis in diseases of the hematopoletic system, neoplastic arthritis, paraneoplastic arthritis, osteoarthritis, arthritis after injuries; arthritis in diseases of articular cartilage, arthritis in neuropathies, arthritis in skin diseases, more particularly psoriasis arthropathy.

Furthermore, mild progressive forms of inflammatory polyarthritis can be distinguished from rheumatoid arthritis.

The methods according to the invention are, in a preferred embodiment, immunological methods. Here, the protein Hdj2 in synovial fluid which was obtained from patients who show an early form of a rheumatic disease is investigated immunologically.

The detection of the protein Hdj2 in synovial fluid is preferably carried out with an antibody, for which, in principle, polyclonal and monoclonal antibodies can be used. Since the amino acid sequence is known (SEQ ID NO:2), it is readily possible to raise antibodies against it For example, rabbits, mice, or rats can be immunized with the human protein Hdj2, if appropriate with the addition of adjuvants. Antiserum of these animals can, preferably in purified form, be used for detecting Hdj2. The spleen cells of the immunized animals can alternatively be fused with tumor cells and the immortalized, monoclonal-antibody-producing cell lines isolated and propagated. After characterizing the epitopes to which the particular monoclonal antibodies can bind, those monoclonal antibodies which deliver the best results in the immunological tests are selected. Alternatively, commercially available monoclonal and polyclonal antibodies can also be used for this purpose.

The immunological characterization of synovial fluid can proceed such that the proteins of the synovial fluid are separated on an electrophoretic gel (SDS-PAGE) and the proteins are then transferred to a suitable membrane (nitrocellulose or nylon). These membranes are then reacted with the antibodies directed against Hdj2, and the bound antibodies are detected with anti-antibodies, wherein the anti-antibodies are generally directed against the Fc region of the anti-Hdj2-specific antibody. The anti-antibodies are usually connected to a color-generating or light-generating enzyme which catalyzes a color-producing reaction or blackens a film.

Alternatively, immunological detection can be carried out for this purpose with ELISA tests. Here, for example, suitable antibodies, preferably monoclonal antibodies, can be attached to the wall of microtiter plates. Subsequently, synovial fluid is added and washed. If the protein Hdj2 is present, it then binds to the ELISA plates. The detection of the protein Hdj2 is then effected with antibodies which are directed against certain epitopes of Hdj2 and which are connected to a color-producing component.

A further embodiment of the present invention relates to test kits with which it is possible to carry out a method according to the invention. Such test kits comprise, as a constituent, an immunological reagent which makes possible the specific detection of the protein Hdj2 in synovial fluid. In a preferred embodiment, the immunological reagents here are monoclonal antibodies which bind specifically and with sufficient sensitivity to suitable epitopes of the protein Hdj2.

The test kits further include customary auxiliaries and consumables which are required for carrying out a method according to the invention. These can be precoated microtiter plates or membranes and also the reagents required for carrying out the test method.

In a particularly preferred embodiment of the present invention, antibodies directed against the protein Hdj2 are used for differential diagnosis of an inflammatory rheumatic disease at an early phase of the disease.

The term “antibody” or “monoclonal antibody”, as used in the present application, is to be understood as also meaning the corresponding binding regions of an antibody. In antibody technology, it has proved to be advantageous to also use monospecific antibody fragments which bind to the desired epitopes, instead of complete antibodies. The term “antibody” can, in the present application, be replaced by Fab fragments, Fv fragments, single-chain antibody fragments (scFv), disulfide-stabilized Fv fragments, V_{H} and camel antibodies; multivalent antibody fragments, bifunctional antibodies, and bispecific antibodies.

Samples of SF were separated by means of SDS-PAGE and stained in an immunoblot with specific antibodies. According to the invention, antibodies against Hdj2 (KA2A5.6) were used. With this immunoblot, bands of the expected molecular weight often appeared.

Further immunohistochemical analyses were also made of synovial tissues (frozen sections), from RA patients and also from control persons with different forms of osteoarthritis (OA), including carpal tunnel syndrome. Use was made of anti-Hdj2 and antibodies with which it was possible with their help to characterize the Hdj2-expressing cells.

The specificity of the antibody was investigated. It was observed that the antibody stains only cells which also contain transcripts of Hdj2 (mRNA of the gene DNAJA1). It was observed that transcripts of Hdj2 are to be found in synovial tissue from RA patients.

The exact diagnoses and all other documents concerning the patients were looked at together. First, the patients were divided into two classes, namely those certain to have RA (diagnosis given, >4 ACR criteria were detectably present) and those certain to have another diagnosis (e.g., reactive arthritis, psoriasis arthritis, etc.). Of a total of 130 patients, 50 had RA, and 80 did not have RA.

Surprisingly, Hdj2 was found in many aspirates.

Subsequently, the diagnoses were checked and further SFs investigated. In the course of the investigation, a significant correlation between the appearance of Hdj2 in SF and the diagnosis RA (p=0.021) emerged. It was also possible to show that Hdj2 was overexpressed immunohistochemically only in sections of synovial tissues from patients with RA, but not in OA.

In the SFs, it was possible to semiquantitatively classify the amount of Hdj2 (0, 1, 2, 3, 4). With this classification and the occurrence as such, an attempt was made to find relationships with other parameters. There was, in RA patients, a positive correlation with the degree of inflammation, determined according to the number of leukocytes infiltrating the joint. In many SFs, rheumatoid factor had already previously been determined in routine laboratories. Anti-CCP antibodies were now additionally determined in all SFs. With both autoantibodies, correlations with the diagnosis RA emerged (in each case, with p<0.001). Correlations were also found between the presence of Hdj2 and both autoantibodies (rheumatoid factor: p=0.038, and anti-CCP: p=0.016). However, there was no complete overlap. Therefore, Hdj2 in SF appears useful as an additional marker, e.g., for forming subgroups.

The question of whether extracellular Hdj2 in RA patients is also to be found in blood also appeared important. Therefore, 14 plasma samples from RA patients were tested, who at the same time point had relatively large amounts of Hdj2 in SF. In these immunoblots, it was not possible to detect Hdj2 in the plasma samples.

Hdj2 in synovial fluid is an early marker of RA. The result of a tissue which originated from a patient with carpal tunnel syndrome and which served as a control for absence of RA was initially incomprehensible. However, the patient file revealed that this patient developed RA more than a year later. There are indications that patients with carpal tunnel syndrome relatively often develop RA. The overexpression of Hdj2 is thus an early sign of early—and still undiagnosed—RA.

Consequently, SF from patients with an unclear diagnosis was also analyzed. In multiple cases in which Hdj2 was already detectable in SF, RA was identified before it was possible to make a diagnosis of RA according to the relevant criteria. Hdj2 is therefore useful as a marker for early recognition of RA and also as a marker for a differential diagnosis.

The examples which follow illustrate the invention.

### EXAMPLE 1

A 53-year-old patient complains of polyarthralgia of the vertebral column and aches in the region of the hand joints. She is investigated internally, neurologically, and rheumatologically. No indication of an inflammatory joint condition is to be found according to current clinical and also serological tests (not even rheumatoid factor). It is explicitly mentioned that, although the patient is HLA-B27 positive, she does not have Bekhterev's disease. The patient is clearly physically stressed by having to care for a severely disabled daughter. Orthopedically, she receives the diagnosis of mild secondary carpal tunnel syndrome, emanating from the vertebral column.

The carpal tunnel syndrome is operated on 7 months later. The synovial tissue removed is investigated in frozen sections with regard to the expression of Hdj2 (and Hsc70). It emerges that both molecules can be detected in this patient at highly elevated levels, which was previously the case only in patients with established RA, while control persons with various other diagnoses (OA) were negative.

One year later, the patient presents to another rheumatologist, with suspected chronic polyarthritis. This diagnosis was made for the first time 2 months earlier during a stay in a health resort. It was established that there is morning stiffness of 3 hours, affliction of the finger joints, symmetrically, and also positive X-ray signs. The diagnosis was now seronegative, ANA-positive, rheumatoid arthritis (with ACR criteria fulfilled).

This example shows the possibility of early recognition of RA in a patient with symptoms of an inflammatory rheumatic disease

### EXAMPLE 2

Synovial fluid was withdrawn from a total of six patients in which the diagnosis of rheumatoid arthritis was clearly confirmed only after appearance of symptoms of a rheumatic disease.

The synovial fluid was separated with the help of SDS-PAGE electrophoresis, and the protein bands were blotted onto nitrocellulose. After blocking nonspecific bindings sites, the nitrocellulose was reacted overnight at 4° C. in a buffer with a primary antibody against Hdj2.

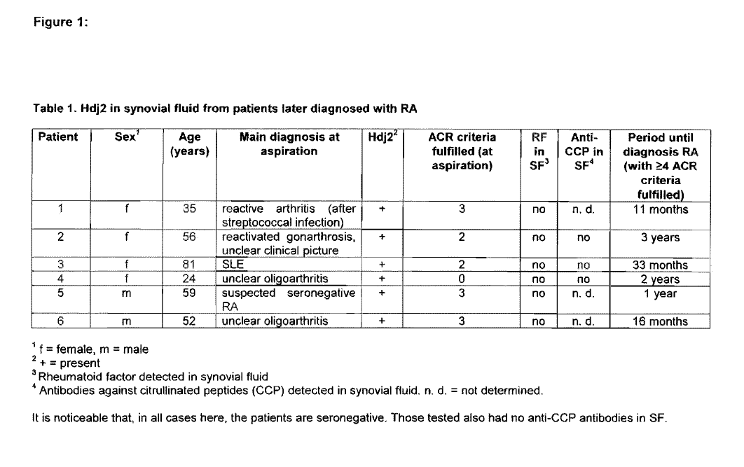

Subsequently, the nitrocellulose was washed three times, and the anti-Hdj2 antibody was detected with the help of an anti-mouse antibody which was raised in goat. Subsequently, the film was developed, and binding was detected semiquantitatively. The results are put together in FIG. 1, which contains table 1.

### EXAMPLE 3

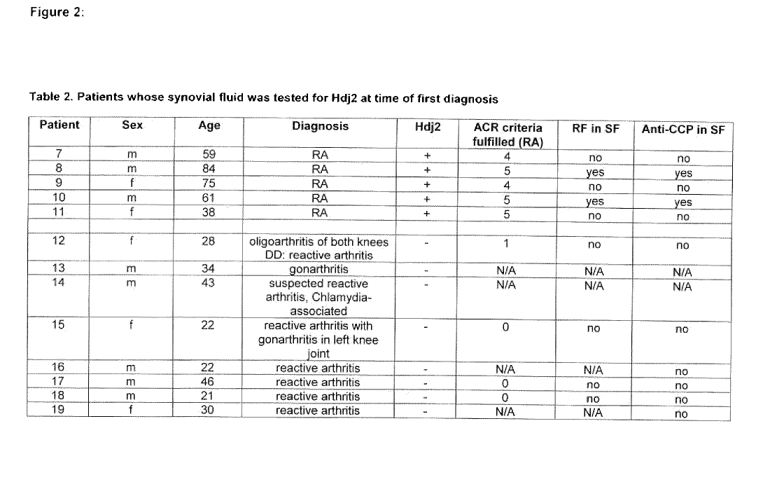

In order to document the time course of the appearance of Hdj2 in synovial fluid, Hdj2 was determined in the synovial fluid of patients who had symptoms for only a few weeks to months, were aspirated for the first time, and received shortly after a reliable diagnosis for the first time. Here, Hdj2 was detected in patients who already fulfilled ACR criteria at this early time. In patients with other distinct diagnoses, Hdj2 was not detected (Patients with undifferentiated arthritis or an unclear diagnosis at this time were not included in this investigation). The measurements were carried out at a later stage of the disease, within a period of between about 0.5 and 1 year after appearance of the first symptoms (or the first diagnosis). The data obtained are listed in FIG. 2, which shows table 2.

### EXAMPLE 4

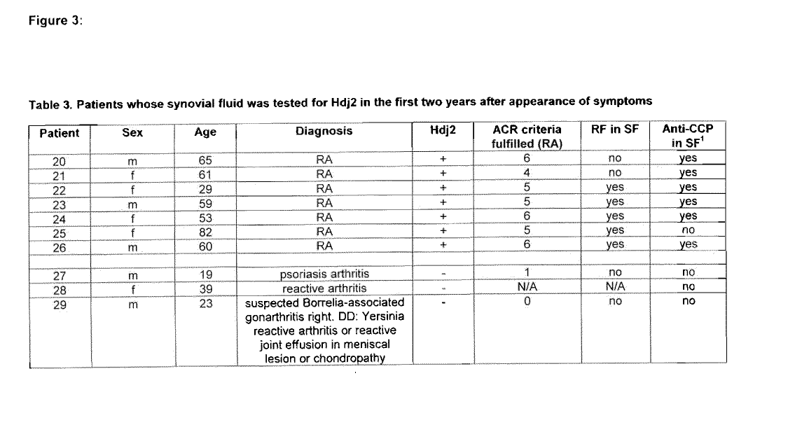

The presence of the Hdj2 protein in synovial fluid was also measured at later times after the appearance of the initial symptoms. In FIG. 3, table 3 shows the data from patients in whom the measurement of Hdj2 in synovial fluid was determined in the first two years after appearance of symptoms. Determination of Hdj2 values was thus carried out within a period of up to 2 years after appearance of the first symptoms (or the first diagnosis). Table 3 provides evidence that all patients who received the diagnosis rheumatoid arthritis (RA) also had Hdj2 protein in synovial fluid. Patients who suffered from rheumatoid diseases showed no Hdj2 protein.

1. In vitro method for determining the type of inflammatory rheumatic disease, wherein the presence or absence of the protein Hdj2 in synovial fluid is determined at an early stage of the disease.

2. The method as claimed in claim 1, wherein the rheumatoid disease is selected from the group consisting of: rheumatoid arthritis, infectious or septic arthritis, arthritis in rheumatic vertebral column diseases; arthritis in inflammatory gut diseases, juvenile chronic arthritis, arthritis in inflammatory connective tissue diseases; allergic arthritis; arthritis caused by metabolic diseases, gout, arthritis in endocrine disorders, hyperthyroidism, hypothyroidism, hyperparathyroidism, hypoparathyroidism, diabetes mellitus, Cushing's syndrome. acromegaly, pheochromocytoma; arthritis in granulomatous diseases; arthritis in joint bleeding as a result of disorders of blood coagulation, arthritis in diseases of the hematopoietic system, neoplastic arthritis, paraneoplastic arthritis, osteoarthritis, arthritis after injuries; arthritis in diseases of articular cartilage, arthritis in neuropathies. arthritis in skin diseases, and psoriasis arthropathy.

3. The method as claimed in claim 1 wherein the presence of the protein Hdj2 in synovial fluid is determined immunologically.

4. The method as claimed in claim 3. wherein the immunological detection method makes use of at least one antibody which binds specifically to an epitope of the protein Hdj2.

5. The method as claimed in claim 3, wherein the method is an ELISA method.

6. The method as claimed in claim 3, wherein the method is a Western blot method.

7. A test kit for carrying out a method as claimed in claim 1. wherein the test kit includes at least one monoclonal antibody which binds specifically to the protein Hdj2.

8. The test kit as claimed in claim 7, wherein the test kit is an ELISA test kit.

9. A method comprising using of monoclonal antibodies against the protein Hdj2 for differential diagnosis of an inflammatory rheumatic disease at the early stage of an inflammatory rheumatic disease, and detecting the antibodies.

10. The method as claimed in claim 2, wherein the presence of the protein Hdj2 in synovial fluid is determined immunologically.

11. The method as claimed in claim 10, wherein the immunological detection method makes use of at least one antibody which binds specifically to an epitope of the protein Hdj2.

12. The method as claimed in claim 10, wherein the method is an ELISA method.

13. The method as claimed in claim 10, wherein the method is a Western blot method.

14. The method as claimed in claim 11, wherein the method is an ELISA method.

15. The method as claimed in claim 11, wherein the method is a Western blot method.
